(19) United States
(12) Patent Application Publication
(10) Pub. No.: US 2005/0177312 A1
(43) Pub. Date: Aug. 11, 2005

(54) Real-time medical data recording system and method

(75) Inventors: Steven E. Guerrant, Durham, NC (US); Stephen P. Foster, Raleigh, NC (US); Cynthia H. Robertson, Durham, NC (US)

Correspondence Address: JENKINS, WILSON & TAYLOR, P. A., 3100 TOWER BLVD, SUITE 1400, DURHAM, NC, 27707, US

(73) Assignee: Duke University

(21) Appl. No.: 10/921,717

(22) Filed: Aug. 19, 2004

Related U.S. Application Data
(60) Provisional application No. 60/496,500, filed on Aug. 20, 2003.

(51) Int. Cl. G06F 17/60; G06F 19/00; G01N 33/48; G01N 33/50

(52) U.S. Cl. 702/19; 705/3; 600/300

(57) ABSTRACT

A medical data recording system and method for real-time storage of medical data received from a plurality of different sources. The system includes a router including an output and a plurality of inputs for connection to a plurality of different medical data sources, and operable to selectively route medical data from one of the plurality of different sources to the output. A medical data recorder serves to record in real-time the medical data from the output of the router to a computer-readable media. A controller is provided and connected to the router for selecting one of the plurality of different medical data sources for route to the output of the router.

### RELATED APPLICATIONS

This application claims the benefit of U.S. Provisional Patent Application Ser. No. 60/496,500 filed Aug. 20, 2003; the disclosure of which is incorporated herein by reference in its entirety.

### TECHNICAL FIELD

The subject matter disclosed herein relates to medical data recording systems and methods. Specifically, the subject matter disclosed herein relates to systems and methods for capturing, displaying, and recording medical data in real-time from a plurality of medical data sources.

### BACKGROUND ART

In the medical field, it is often necessary to capture, display, and store certain medical data when examining or performing a procedure on a patient. Such data can be collected from a variety of different sources such as an analog or digital video camera or still camera for capturing audio/visual information. Additionally, medical data can be gathered from medical modalities such as sophisticated radiology equipment grouped as small matrix size and large matrix size instruments. Small matrix systems include equipment for magnetic resonance imaging (MRI), computed tomography (CT), ultrasonography (US), nuclear medicine (NM) and digital fluorography. Large matrix systems include equipment for computer radiography (CR) and digitized radiography (DR). Other data image acquisition equipment may be used for radiofluoroscopy, angiography, such as x-ray angiography and heart scanning. Still other equipment of great usefulness in acquiring medical information includes secondary capture devices for endoscopy, microscopy, and photography, such as scanners, and electrocardiogram (ECG) machines.

The resulting medical data may take numerous forms, including text, images and video, or variations thereof, such as image overlay data, measurements, coordinates, etc. Medical data may also be in the form of time-dependent data including sound, such as audio dictation, and waveform data. The data may be static representations of time dependent forms, such as curves. Thus, it is advantageous for medical data recording systems that may need to store and display medical data to be flexible, so as to accommodate this variety of data from multiple sources.

### SUMMARY

According to one aspect, a medical data recording system for real-time storage of medical received from a plurality of different sources is provided. The system can include a router including an output and a plurality of inputs for connection to a plurality of different medical data sources, and operable to selectively route medical data from one of the plurality of different sources to the output. The system can also include a medical data recorder operable to record the medical data from the output of the router to a computer-readable media. Further, the system can include a controller connected to the router for selecting one of the plurality of different medical data sources for route to the output of the router.

According to a second aspect, a method for selectively storing medical data received from a plurality of different sources in real-time is provided. The method can include receiving medical data from a plurality of different medical data sources. The method can also include selecting one of the plurality of different medical data sources. Further, the method can include recording medical data from the selected medical data source.

Accordingly, it is an object of the subject matter disclosed herein to provide a medical data recording system.

It is another object of the subject matter disclosed herein to provide for real-time storage of medical received from a plurality of different sources.

Some of the objects having been stated hereinabove, and which are achieved in whole or in part by the present subject matter, other objects will become evident as the description proceeds when taken in connection with the accompanying drawings as best described hereinbelow.

### BRIEF DESCRIPTION OF THE DRAWINGS

Exemplary embodiments of the subject matter disclosed herein will now be explained with reference to the accompanying drawings, of which:

FIG. 1 is a schematic view of an exemplary medical data recording system including various medical data sources for capturing medical data during a medical examination or procedure and recording the medical data on a computer-readable media; and

FIG. 2 is a flow chart illustration of a process for selectively storing medical data received from a plurality of different medical data sources in real-time according to an embodiment of the subject matter disclosed herein.

### DETAILED DESCRIPTION

Systems and methods for selectively capturing, displaying, and recording medical data in real-time from one of a plurality of different sources are disclosed herein. The systems and methods according to the subject matter disclosed herein will be explained in the context of a flow chart and diagram. It is understood according to the subject matter disclosed herein that the flow chart and diagram can be implemented in hardware, software, or a combination of hardware and software. Thus, the subject matter disclosed herein can include computer program products comprising computer-executable instructions embodied in computer-readable media for performing the steps illustrated in each of the flow charts or implementing the machines illustrated in each of the diagrams. In one embodiment, the hardware and software for selectively capturing, displaying, and recording medical data on computer-readable media from one of a plurality of different sources in real-time are located in client and/or server computers having communication via a network connection. Alternatively, the hardware and software for selectively capturing, displaying, and recording medical data on computer-readable media from one of a plurality of different sources in real-time can be located in a single stand-alone or general purpose computer.

Medical data sources according to the subject matter disclosed herein may include any device for capturing medical data in electronic form, such as devices which capture data during a medical operation or procedure and generating output in electronic form. Some common formats include video, images, sound, such as audio dictation, text, waveform, curves, and/or combinations or variations thereof. Additional video formats can include MPEG2 and MPEG4 video/audio files, video transmission formats can include S-video, Y/C, SDI, and DVI-I. The network protocol video format can be DICOM. Although the data as described herein relates to the medical field, the data may also be applied to any number of fields, such as manufacturing, veterinary science, and scientific research.

Medical data may be grouped into various types. For example, clinical data is information acquired by a medical modality during the examination of a patient and relates to the patient's physical health. Examples of clinical data may include radiology images, camera photographs, sound recordings, and the like. Parameter data is a type of data that represents criteria surrounding the acquisition of clinical data. Parameter data includes the settings of the medical modality acquiring the clinical data, relationships of multiple sets of data such as overlay data, timing of the data acquisition, measurements, coordinates, and the like. The parameter data includes some of the information required by DICOM standards (as originally published by an ACR-NEMA committee sponsored by the American College of Radiology and the National Electrical Manufacturers Association as Digital Imaging and Communications in Medicine (DICOM), NEMA Publications PS 3.1-PS3.12, by The National Electrical Manufacturers Association, Rosslyn, Va., 1992, 1993, 1994, 1995) for stored and transferred medical files. Other medical data may include 3-D volume data, series data for all clinical data in a medical series, annotation data for notes made by a practitioner, and background data such as patient history and/or physical examination information.

Medical data can be captured by many different types of medical data sources in accordance with the subject matter disclosed herein. Medical sources can include still cameras and digital or analog video cameras. Additionally, medical sources or modalities can include equipment for angiography, radiography, endoscopy, microscopy, physical exams and waveform devices to collect EEG and/or ECG data, such as from Hewlett Packard Corporation of Palo Alto, Calif., Datex-Ohmeda of Madison, Wis., GEMS of Jupiter, Fla., Karl Storz of Culver City, Calif., Carl Zeiss Inc. of Thornwood, N.Y., and Olympus America of Melville, N.Y.

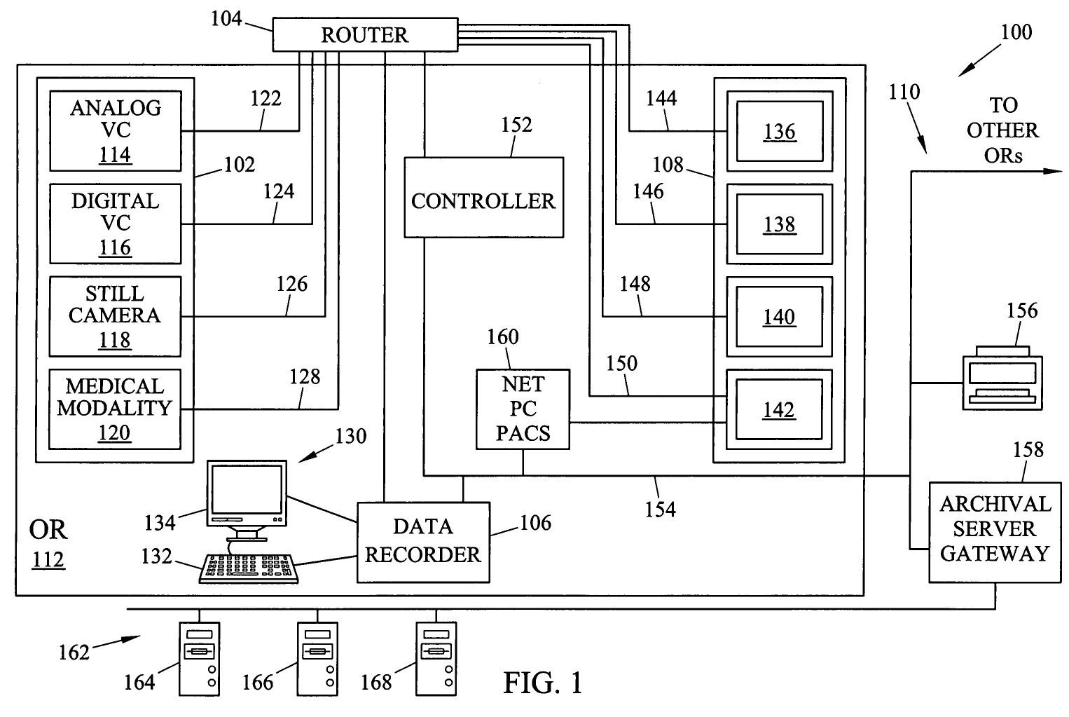

Referring to FIG. 1, an exemplary medical data recording system, generally designated 100, is illustrated including various medical data sources 102 for capturing medical data during a medical examination or procedure and recording the medical data on a computer-readable media. System 100 can include a router 104 having various inputs for connection to medical data sources 102 for routing captured medical data to a data recorder 106, various displays 108, and a local area network (LAN), generally designated 110. Router 104 can include various outputs for operable connection to data recorder 106, displays 108, and LAN 110. In this embodiment, medical data sources 102, data recorder 106, displays 108, and a connector for LAN 110 are located in an operating room (OR) 112. Router 104 is located outside of OR 112. Alternatively, router 104 can be located in OR 112.

Medical data sources 102 can capture medical data during a medical procedure or examination. In this embodiment, medical data sources 102 can include an analog video camera 114, a digital video camera 116, a still camera 118, and a medical modality 120, such as an X-ray computed tomography (CT) unit, a magnetic resonance imaging (MRI) unit, an ultrasonic diagnostic unit, an X-ray photographing unit, endoscopy rigid and flexible scope, microscopy equipment, fluoroscopy equipment, physiologic monitoring equipment, and various cameras. In this embodiment, router 104 is connected to analog video camera 114 and digital video camera 116 via a Y/C (Luminance/Chrominance) video cable 122 and an SDI (Serial-Digital Interface) cable 124, respectively. Still camera 118 and medical modality 120 can be connected to router 104 via an SDI cable 126 and a line 128, respectively. The fluoroscopy and ultrasound can connect to the router through an analog composite cable.

Router 104 can switch the selected input to any of the selected outputs. Additionally, the router can determine if an input signal is present and pass that information on to the control computer processing unit (CPU). The router can condition the video signal that it receives before sending it on.

System 100 can also include input/output devices, generally designated 130, for displaying information to an operator and receiving input information from the operator. In this embodiment, input/output devices 130 can include a keyboard 132 and a touch screen display 134. Alternatively, input/output devices 130 can include a mouse, a conventional monitor, or any other suitable input/output devices known to those of skill in the art. Furthermore, input/output devices 130 can include a speaker and software/hardware operable to receive and interpret voice commands from an operator. Input/output devices 130 are operable for transmitting control information and receiving information. Input/output devices 130 can be control and monitor data recorder 106 and router 104 via a connection through data recorder 106.

An operator can input information into input/output devices 130 for displaying data, recording data, and controlling medical data sources 102. In the area of video capture, the operator can input information for beginning a new case, start recording data, stop recording data, capturing a still image, adjusting image quality, printing, finalizing the disk, selecting a video source, and exporting still images for archive. In the area of video routing, the operator can input information for selecting the display to route an image, selecting the source to transmit a display, and selecting a source from another room to display. In the area of application selection, the operator can input information for selecting the application for action. In the area of PACS retrieval, the operator can input information for logging into the system, selecting patient pre-fetched images, image selection, orientation, overlay, and magnify.

In this embodiment, displays 108 can include cathode ray tube (CRT) monitors 136, 138, and 140 and a flat panel monitor 142 for displaying the medical data converted by medical data sources 102. Alternatively, displays 108 can include any suitable display known to those of skill in the art. Router 104 can route the information from analog video camera 114, still camera 118, medical modality 120, and digital video camera 116 to monitor 136, 138, 140, and 142, respectively. An operator can enter command information into input/output devices 130 for selectively routing medical data from one of medical data sources 102 to one of displays 108. Monitors 136, 138, and 140 can be connected to router 104 via coaxial cables 144, 146, and 148, respectively. Flat panel display monitor 142 can be connected to router 104 via a cable 150 via a 5 conductor coaxial cable. In this embodiment, monitor 142 is a digital flat panel monitor having a 1600-by-1200 pixel screen resolution and produced by National Display Systems, Inc. of Morgan Hill, Calif. Additionally, this monitor can be cabled via twin coaxial cables for analog video display.

Data recorder 106 can record medical data from one of medical data sources 102 via selective routing through router 104. In this embodiment, medical data is recorded on an optical disk, such as a compact disc or DVD. Data recorder 106 can save still images on command and save them to the optical disk or send them to be printed. The still images can be arranged on a printed page as a single image or grouped in even numbers up to 8 per page. Alternatively, medical data can be recorded on another suitable computer-readable media known to those of skill in the art, such as a floppy disk or a computer hard drive. An operator can input information into input/output devices 130 for controlling router 104 to route the medical data from one of medical data sources 102 to data recorder 106. Additionally, the operator can input command information for controlling data recorder 106 to record and edit different portions of the received medical data. After recording medical data relating to a patient, the computer-readable media having the recorded medical data can be placed in the patient's file history. The recorded files can be formatted in MPEG2, MPEG4, or DIACOM.

Router 104 and data recorder 106 can be connected to the components of LAN 110 via a network controller 152 and network connection cable 154. Controller 152 can be a computer operable to receive commands from input/output device 130. Based on the received commands, controller 152 can transmit commands to router 104, displays 108, and other components of system 100. According to one embodiment, network controller 150 is a Crestron computer that interprets commands it receives from 134 and 132 and in turn sends out commands to components 104, 110, 106, and 160 (described in further detail below) which is available from Crestron Electronics, Inc. of Rockleigh, N.J., U.S.A. The network connection can comprise an Ethernet connection. Other communication schemes are suitable for transfer of medical data from router 104 and data recorder 106 to the components of LAN 110, such as serial interfaces, parallel interfaces, RS422 and/or RS432 interfaces, Livewire interfaces, IEEE-1394 serial busses, Appletalk busses, ATM busses and/or networks, token ring and/or other local area networks, universal serial busses, PCI buses, and wireless (e.g., infrared) connections.

LAN 110 can include a printer 156 and a server gateway 158. In addition, LAN 110 can include other suitable network devices known to those of skill in the art. Medical data captured from sources 102 and medical data recorded by data recorder 106 can be transmitted to server gateway 158 for formatting and compression. Server gateway 158 can be a computer system that stores medical data is accessible through a network, e.g., the Internet, an intranet, or an extranet. Server gateway 158 can have a direct connection to other medical image servers across the system and can poll them for any images pertaining to the patient. These images can be pre-fetched and available for display at the surgeon's request. Images recorded in the OR can be sent to the gateway server for compression and long term archival storage. Additionally, the captured and recorded medical data can be transmitted to printer 156 for printing the medical data. In this embodiment, printer 156 is a high resolution printer for printing high quality medical images. Both color and black and white images of all types can be printed.

Medical data can also be transmitted to workstations attached to LAN 110 for review and edit by an operator. Such workstations can also access server gateway 158 for retrieving stored medical data.

System 100 can also include a net PACS PC 160 for connecting flat panel monitor 142 to LAN 110. PC 160 can be a computer operable to implement an application for acquiring and displaying medical images, such as PACS images, from any accessible server. PC 160 can poll accessible servers storing medical images based on a patient's medical record number, and acquire all recorded images on the server. Such images can include radiographic, ultrasound, MRI, CT, Cath Lab, endoscopic, C-arm, vascular, microscopic, and pathology digital images.

Medical data recording system 100 can also include a wide area network (WAN), generally designated 162. WAN 162 can include a radiology PACS server 164, a MRI PACS server 166, and a Cath Lab PACS server 168. These servers store images created from the use of ionizing radiation and magnetic resonance in a variety of modalities.

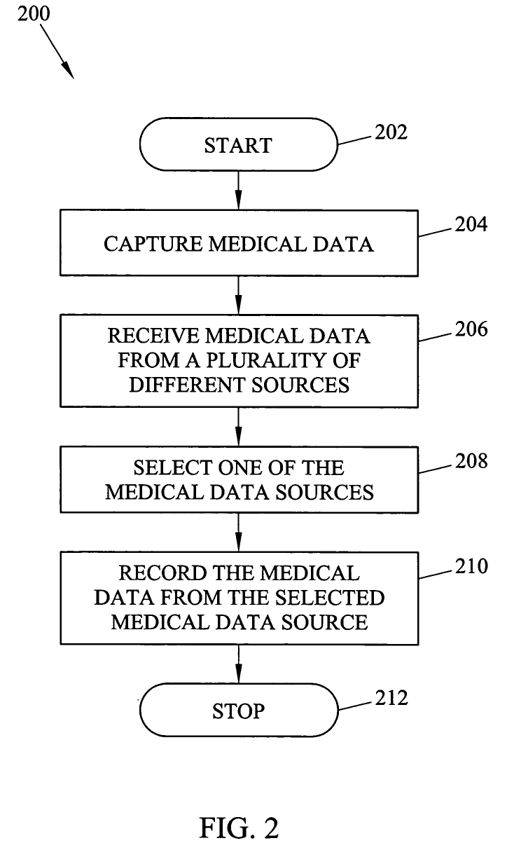

Referring to FIG. 2, a flow chart, generally designated 200, is provided which illustrates a process for selectively storing medical data received from a plurality of different medical data sources in real-time according to an embodiment of the subject matter disclosed herein. FIG. 2 is discussed below in connection with FIG. 1, described in detail above. The process begins at the step indicated by reference numeral 202. Next, the different medical data sources 102 are operated to capture medical data during a medical examination or procedure (step 204). At step 206, router 104 can receive the captured medical data via lines 122, 124, 126, and 128. Next, the operator can control controller 104 to select one of the medical data sources via input/output device 130 for routing to data recorder 106, displays 108, LAN 110, or WAN 162 (step 208). The medical data from the selected medical data source can then be routed to data recorder 106 for recording on a computer-readable media (step 210). The process then stops (step 212).

It will be understood that various details of the subject matter disclosed herein may be changed without departing from the scope of the subject matter disclosed herein. Furthermore, the foregoing description is for the purpose of illustration only, and not for the purpose of limitation.

1. A medical data recording system for real-time storage of medical data received from a plurality of different sources, the system comprising:
(a) a router including an output and a plurality of inputs for connection to a plurality of different medical data sources, and operable to selectively route medical data from one of the plurality of different sources to the output;
(b) a medical data recorder operable to record in real-time the medical data from the output of the router to a computer-readable media; and
(c) a controller connected to the router for selecting one of the plurality of different medical data sources for route to the output of the router.

2. The medical data recording system of claim 1 further including more than one output and operable to selectively route medical data from one of the plurality of different medical data sources to more than one output.

3. The medical data recording system of claim 2 further including at least one display connected to one of the outputs for displaying medical data from one of the plurality of different medical data sources.

4. The medical data recording system of claim 3 wherein the at least one display comprises a monitor.

5. The medical data recording system of claim 4 wherein the monitor comprises a cathode-ray tube monitor.

6. The medical data recording system of claim 4 wherein the monitor comprises a flat panel monitor.

7. The medical data recording system of claim 2 further including at least one server computer connected to one of the outputs of the router for storing the medical data received from one of the outputs.

8. The medical data recording system of claim 7 wherein the at least one server includes a web browser operable for access via the Internet.

9. The medical data recording system of claim 7 wherein the at least one server computer comprises a picture archiving and communications systems server.

10. The medical data recording system of claim 7 wherein the at least one server computer is connected to the output of the router by a network connection.

11. The medical data recording system of claim 1 wherein the medical data sources include a video imaging device.

12. The medical data recording system of claim 1 wherein the medical data sources include a still image device.

13. The medical data recording system of claim 1 wherein the data recorder comprises an optical disk writer.

14. The medical data recording system of claim 13 wherein the optical disk writer is operable to write computer-readable data to a compact disc.

15. The medical data recording system of claim 13 wherein the optical disk writer is operable to write computer-readable data to a DVD.

16. The medical data recording system of claim 1 wherein the data recorder is operable to record video and audio data.

17. The medical data recording system of claim 1 wherein the data recorder is operable to record still images.

18. The medical data recording system of claim 1 wherein the controller comprises an operator input/output device.

19. The medical data recording system of claim 1 wherein the controller comprises a touch screen display.

20. The medical data recording system of claim 1 wherein the medical data sources include a video capture device.

21. The medical data recording system of claim 21 wherein the video capture device includes a video camera.

22. A method for selectively storing medical data received from a plurality of different medical data sources in real-time, the method comprising:
(a) receiving medical data from a plurality of different medical data sources;
(b) selecting one of the plurality of different medical data sources; and
(c) recording medical data in real-time from the selected medical data source.

23. The method of claim 22, wherein the medical data sources include a video imaging device.

24. The method of claim 22, wherein the medical data sources include a still image device.

25. The method of claim 22, wherein the data recorder comprises an optical disk writer.

26. The method of claim 25, wherein the optical disk writer is operable to write computer-readable data to a DVD.

27. The method of claim 22, wherein the data recorder is operable to record video and audio data.
